(19) United States
(12) Patent Application Publication
(10) Pub. No.: US 2026/0287698 A1
(43) Pub. Date: Sep. 24, 2026

(54) Recognition Based Medical Imaging System: Methods and Systems for Enhanced Medical Image Acquisition and Reconstruction Using Recognition Physics

(71) Applicant: Jonathan Washburn, Austin, TX (US)

(72) Inventor: Jonathan Washburn, Austin, TX (US)

(21) Appl. No.: 19/296,734

(22) Filed: Aug. 11, 2025

Related U.S. Application Data
(60) Provisional application No. 63/771,419, filed on Mar. 13, 2025.

(51) Int. Cl. G01R 33/56; A61B 6/00; A61B 8/00; G01R 33/48

(52) U.S. Cl. CPC G01R 33/5608; A61B 6/5247; A61B 8/5261; G01R 33/481; G01R 33/4814

(57) ABSTRACT

A recognition-based medical imaging framework is disclosed that computes a bounded recognition coverage value from a quality signal under a scaling parameter Xopt, where Xopt is set to phi over pi or calibrated, and uses the same value to drive real-time acquisition control and reconstruction weighting, the modality-agnostic system for computed tomography magnetic resonance imaging ultrasound positron emission tomography and digital radiography runs on CPU GPU or FPGA within vendor safety limits and achieves reduced dose or scan time at matched image quality or improved signal-to-noise or contrast-to-noise at matched dose with outputs recorded in standards-compliant images and metadata.

### CROSS-REFERENCE TO RELATED APPLICATIONS

This application claims priority under 35 U.S.C. § 119(e) to U.S. Provisional Patent Application No. 63/771,419, filed on Mar. 13, 2025, titled “Recognition-Based Medical Imaging System: Methods and Systems for Enhanced Medical Image Acquisition and Reconstruction Using Recognition Physics,” the entire contents of which are incorporated herein by reference.

### STATEMENT REGARDING FEDERALLY SPONSORED RESEARCH OR DEVELOPMENT

No federal funding or government contract supported the research or development disclosed in this application.

### NAMES OF THE PARTIES TO A JOINT RESEARCH AGREEMENT

Not applicable.

### REFERENCE TO A SEQUENCE LISTING, A COMPUTER PROGRAM LISTING, OR A LARGE TABLE APPENDIX

Not applicable.

### BACKGROUND OF THE INVENTION

Medical imaging systems—including magnetic resonance imaging (MRI), computed tomography (CT), ultrasound, positron emission tomography (PET), and digital radiography (DR/X-ray)—are central to modern diagnosis and therapy planning. Despite significant progress in detector technology, acquisition control, and reconstruction algorithms, these modalities still face persistent trade-offs among radiation or energy exposure, acquisition time, motion sensitivity, image noise, artifact burden, and computational latency.

In CT and DR/X-ray, image quality often improves with increased tube current or exposure time, but higher radiation dose elevates patient risk. Vendors employ automatic exposure control and tube-current/kVp modulation to mitigate dose, yet these methods are typically heuristic, scanner-specific, or anatomy-templated and may not adapt at sufficiently fine spatial or temporal scales to patient-specific variation, motion, or contrast distribution throughout an exam.

In MRI, sequence design (e.g., TR/TE/flip angle), sampling trajectories, and acceleration strategies (e.g., parallel imaging and compressed sensing) can reduce scan time and improve resolution. However, selecting these parameters remains a complex, multi-objective process influenced by hardware constraints, patient physiology, and pathology. Existing adaptive or model-based approaches often optimize for limited criteria or single sequences and may not generalize across anatomies, field strengths, and hardware vendors without extensive manual tuning.

In ultrasound, beamforming, time-gain compensation, adaptive focusing, and speckle-suppressing post-processing improve contrast and lesion conspicuity. Nevertheless, conventional pipelines can struggle to distinguish low-contrast structures in challenging acoustical environments, and dynamic adaptation is frequently constrained by device throughput, simplistic feedback metrics, or operator-dependent presets.

In PET, iterative reconstruction methods (e.g., OSEM variants with priors) and acquisition-time optimization aim to elevate signal-to-noise ratio (SNR) while limiting patient dose and scanner occupancy. These solutions can be sensitive to regularization choices, convergence tuning, and count statistics, and they may not leverage patient-specific informational structure available during acquisition to guide time allocation or reconstruction weighting.

Across modalities, advanced iterative and learning-based reconstructions (e.g., penalized likelihood, dictionary/transform sparsity, and deep-learning denoisers) can deliver material gains in noise-resolution trade-offs. However, such improvements are typically realized post-acquisition and may not feed back into the live acquisition loop to modulate energy delivery, trajectory selection, or sampling density in real time. Moreover, learned methods can be sensitive to dataset bias and require careful curation, calibration drift monitoring, and uncertainty handling to maintain clinical reliability.

A unifying limitation of current practice is the absence of a principled, modality-agnostic framework that: (i) quantifies the informational coherence present in measured data at the point of acquisition; (ii) uses that quantification to adapt acquisition parameters, sampling, and energy delivery in real time for the individual patient; and (iii) carries the same quantification consistently into reconstruction to guide weighting, regularization, and filtering. Existing pipelines frequently treat acquisition control and image reconstruction as loosely coupled stages, leaving potential performance on the table.

There is thus a need for systems and methods that provide a rigorous, quantifiable measure of information coverage or coherence that can be computed on-the-fly, is stable across anatomies and devices, and is simple enough to implement on embedded or accelerated hardware (e.g., FPGA/GPU) with tight latency budgets. Such a measure should drive both upstream acquisition control (dose/current/sequence/trajectory/beamforming) and downstream reconstruction (frequency-domain weighting, spatial regularization, and voxel-wise filtering) in a coordinated way.

Recognition Physics introduces a mathematically grounded approach in which an optimal informational scaling parameter (denoted herein as Xopt and, in certain embodiments, defined by the ratio phi over pi) and an associated recognition coverage function (denoted herein as Fcov) quantify the degree to which measured signals are informationally coherent relative to noise and modeling uncertainty. While specific formulae and implementations are provided elsewhere in this specification, the salient point for the background is that Xopt and Fcov furnish a compact, modality-agnostic control signal that can be evaluated rapidly and used consistently across acquisition and reconstruction.

In acquisition, a recognition-coverage-driven controller can, for example: (i) modulate tube current and kVp in CT/DR/X-ray; (ii) select or adapt MRI sequence parameters (e.g., TR/TE/flip angle) and k-space sampling density; (iii) adjust ultrasound beamforming weights and time-gain compensation; and (iv) allocate PET acquisition time or apply count-domain weighting. In reconstruction, the same coverage measure can guide frequency-space filters, spatially varying regularization, or voxel-wise weighting to improve SNR and resolution while preserving diagnostically important features.

Because Xopt and Fcov are defined by a small number of stable quantities, they can be computed under strict latency (sub-frame or sub-projection) constraints and deployed on practical accelerators. This enables closed-loop behavior in which intermediate coverage estimates inform immediate acquisition adjustments and then carry forward into the reconstruction stage without inconsistencies between what was measured and how it is processed.

The medical imaging industry—exceeding forty-five billion dollars annually—would benefit from a unified, recognition-based framework that (i) reduces radiation or energy exposure without sacrificing diagnostic confidence; (ii) improves resolution and contrast-to-noise in anatomies and pathologies that challenge existing heuristics; (iii) shortens or stabilizes scan time via smarter sampling; and (iv) integrates with existing scanner hardware and clinical workflows through well-defined interfaces.

The present disclosure addresses these needs by providing systems and methods that compute patient- and context-specific recognition coverage during acquisition and apply the same coverage consistently in reconstruction. By establishing a common informational currency across modality control and image formation, the disclosed technology advances beyond heuristic dose/parameter presets and after-the-fact denoising, enabling principled, real-time, closed-loop optimization that is compatible with current clinical infrastructure.

### SUMMARY OF THE INVENTION

The invention provides a recognition-based framework for medical imaging that unifies real-time acquisition control and image reconstruction through a common informational signal called recognition coverage. A recognition scaling parameter, Xopt (in certain embodiments set to the ratio phi over pi or determined by an optimization on historical or live data), and a recognition coverage function, Fcov(r, Xopt), quantify informational coherence of measured signals r and are computed rapidly during acquisition. The same Fcov is then used downstream in reconstruction to guide frequency-domain weighting, spatially varying regularization, and voxel-wise filtering, producing improved resolution and contrast-to-noise while enabling dose or energy reductions and maintaining clinical throughput.

In one aspect, a method comprises: (i) receiving raw acquisition data and associated scanner/patient metadata; (ii) computing one or more live quality metrics (including but not limited to signal-to-noise ratio, local contrast, count statistics, motion or artifact indicators) and deriving a recognition coverage value Fcov for the current context using Xopt; (iii) adaptively selecting or modulating acquisition parameters in a closed loop based on Fcov (for example, exposure settings, sequence timing, sampling density, beamforming, or dwell time); (iv) reconstructing images using the same Fcov to weight filters and regularizers in the projection, k-space, frequency, or image domain; and (v) outputting DICOM images and companion metadata that record acquisition and reconstruction settings and coverage statistics.

In another aspect, a system comprises: an imaging sensor assembly (for CT/DR, an X-ray tube and detector; for MRI, RF coils and gradients; for ultrasound, a transducer array; for PET, scintillation detectors); an acquisition controller configured to compute recognition coverage in real time and to drive modality-specific parameters; and a reconstruction engine configured to incorporate the same coverage values into reconstruction weighting. The controller and reconstruction engine may execute on CPU/GPU and/or FPGA hardware under sub-projection, sub-frame, or view-by-view latency constraints and communicate with scanner subsystems through vendor-supported APIs and safety interlocks.

In another aspect, a non-transitory computer-readable medium stores instructions that, when executed by one or more processors, cause the processors to perform the method of paragraph [0020], including computing Fcov(r, Xopt) and applying it both to acquisition control and to reconstruction weighting.

In certain embodiments, Fcov(r, Xopt) is defined as r divided by (r plus Xopt), providing a stable, bounded weight that increases with coherent signal and decreases with noise or uncertainty. In some embodiments, Xopt is set to phi divided by pi; in other embodiments, Xopt is estimated by maximizing an information criterion or fitted from calibration or prior studies. The framework is modality-agnostic and can accept alternative monotone coverage functions that preserve the closed-loop behavior described herein.

Modality-specific embodiments include: (i) CT/DR: view- or region-specific tube-current and kVp modulation driven by Fcov, and reconstruction with Fcov-weighted filters or iterative penalties; (ii) MRI: adaptive selection of TR/TE/flip angle and sampling density in k-space based on coverage maps, with reconstruction using Fcov-weighted parallel/iterative or compressed-sensing pipelines; (iii) ultrasound: dynamic beamforming, time-gain compensation, and speckle-aware post-processing guided by coverage, with reconstruction and envelope/log compression applying Fcov weights; (iv) PET: acquisition-time allocation or count-domain weighting by coverage and OSEM-type reconstruction with Fcov-informed priors.

In some embodiments, coverage maps are computed per projection, per k-space tile, per receive channel, per time bin, or per voxel, and are smoothed or regularized to ensure controller stability. Quality metrics used to derive r may include local signal-to-noise ratio, gradient energy, residuals from a forward model, motion or physiological phase indicators, and detector health signals. The controller may enforce safety and stability constraints (rate limits, bounds, vendor interlocks) and revert to manufacturer defaults when confidence is low.

The reconstruction engine applies the same coverage values to harmonize acquisition and image formation. Examples include: frequency-domain filters whose passband weights are multiplied by Fcov; iterative solvers with spatially varying penalties scaled by Fcov; and voxel-wise post-filters where each voxel is multiplied by Fcov of a local signal-to-noise or contrast measure. Using a shared coverage signal across the pipeline reduces inconsistency between what is measured and how it is processed.

The framework integrates with existing scanners via software interfaces and preserves clinical workflow: it reads and writes DICOM; logs controller decisions, coverage summaries, and parameter histories for audit and regulatory traceability; and supports configuration by anatomy, protocol, and site policy. Implementations may employ CPU/GPU kernels and/or FPGA logic to meet latency targets and can be deployed as a software upgrade or as an adjunct compute module.

Advantages include dose or energy reductions while preserving or improving image quality, increased effective resolution and contrast-to-noise in challenging anatomies, stabilized or shortened scan times via smarter sampling, and improved reproducibility across patients and devices. In various implementations, the system achieves substantial reductions in exposure at comparable diagnostic confidence and measurable gains in resolution-normalized noise metrics, while maintaining throughput compatible with clinical practice.

### BRIEF DESCRIPTION OF THE DRAWINGS

FIG. 1 is a system block diagram (100) showing acquisition controller (110), modality sensor subsystem (112), reconstruction engine (120), compute module (130), vendor interface (150), storage/PACS (160), operator console (170), and safety interlocks (180).

FIG. 2 is a data-flow diagram (200) showing raw data ingress (202), metadata/sensor signals (204), recognition-coverage computation (210), controller decisions (212), reconstruction pathway (220), DICOM egress (230), and audit logs (240).

FIG. 3 is a modality-agnostic closed-loop acquisition flowchart (300) showing measure (302), compute coverage (304), decide/update parameters (306), apply bounds/interlocks (308), and continue acquisition (310).

FIG. 4 is a reconstruction pipeline diagram (400) showing frequency-domain weighting (410), spatially varying regularization (412), voxel-wise post-weighting (414), coverage-map ingestion (418), and DICOM writer (422).

FIG. 5 is a timing and latency diagram (500) showing measurement window (502), coverage compute (504), decision window (506), actuator update (508), latency budget (510), and rate limiter/interlock boundary (520).

FIG. 6 is an equation FIG. (600) depicting the recognition-coverage function Fcov(r, Xopt) rendered as a drawing and annotated with signal input r (602) and coverage output (604).

FIG. 7 is an equation FIG. (700) depicting the recognition scaling parameter Xopt defined, in certain embodiments, as phi divided by pi, rendered as a drawing and annotated with phi (702) and pi (704).

FIG. 8 is an equation FIG. (800) depicting an example reconstruction weighting expression rendered as a drawing and annotated with frequency-domain weights (802), spatial penalty scale (804), and voxel-wise weight (806).

FIG. 9 is a comparative output panel (900) showing representative phantom or clinical images before (902) and after (904) recognition-based acquisition and reconstruction, alongside quantitative metrics (906).

### REFERENCE NUMBERS LIST

100—System block
110—Acquisition controller
112—Modality sensor subsystem
120—Reconstruction engine
130—Compute module
150—Vendor interface
160—Storage/PACS
170—Operator console
180—Safety interlocks
200—Data-flow diagram
202—Raw data ingress
204—Metadata/sensor signals
210—Recognition-coverage computation
212—Controller decisions
220—Reconstruction pathway
230—DICOM egress
240—Audit logs
300—Closed-loop acquisition flowchart
302—Measure
304—Compute coverage
306—Decide/update parameters
308—Bounds/interlocks
310—Continue acquisition
400—Reconstruction pipeline
410—Frequency-domain weighting
412—Spatially varying regularization
414—Voxel-wise post-weighting
418—Coverage-map ingestion
422—DICOM writer
500—Timing/latency diagram
502—Measurement window
504—Coverage compute
506—Decision window
508—Actuator update
510—Latency budget
520—Rate limiter/interlock boundary
600—Equation figure: Fcov(r, Xopt)
602—Signal input r
604—Coverage output
700—Equation figure: Xopt definition
702—phi
704—pi
800—Equation figure: reconstruction weighting expression
802—Frequency-domain weights
804—Spatial penalty scale
806—Voxel-wise weight
900—Comparative panel
902—Before image
904—After image
906—Quantitative metrics

### DETAILED DESCRIPTION OF THE INVENTION

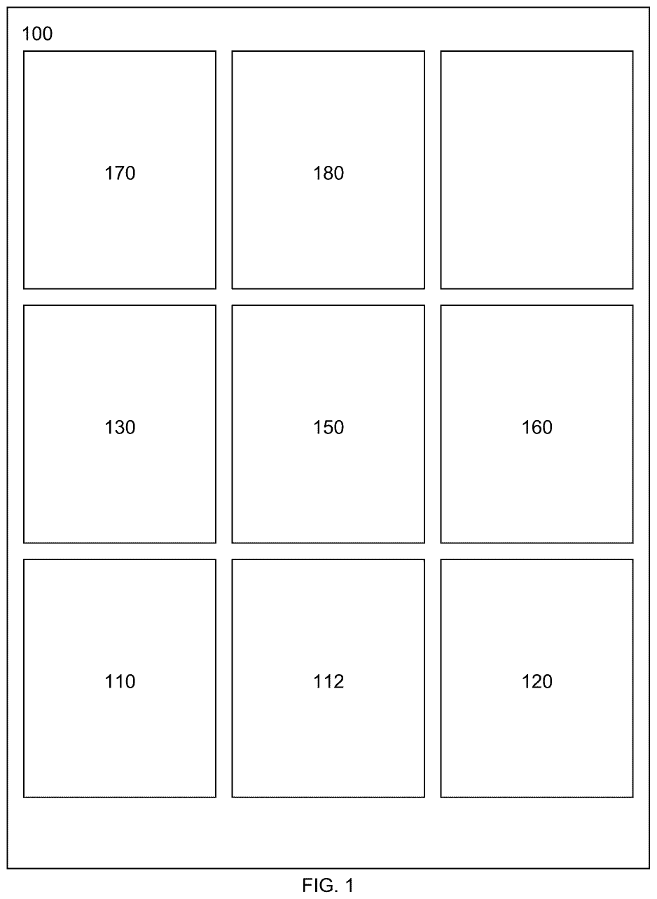

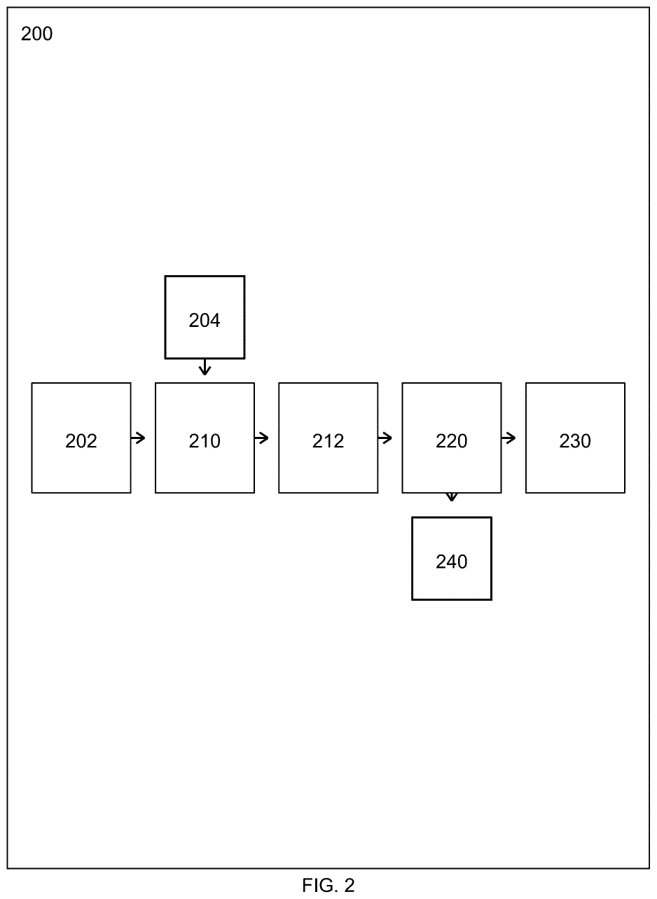

The embodiments described herein provide a recognition-based framework that unifies real-time acquisition control and image reconstruction using a shared informational signal called recognition coverage. The system block diagram is shown in FIG. 1 (100) and the end-to-end data flow is shown in FIG. 2 (200). The framework is modality-agnostic and applies to magnetic resonance imaging, computed tomography, ultrasound, positron emission tomography, and digital radiography without loss of generality.

System architecture. Referring to FIG. 1 (100), the acquisition controller (110) interfaces with a modality sensor subsystem (112) via a vendor interface (150). The controller (110) executes recognition-coverage computation and closed-loop parameter updates, while the reconstruction engine (120) ingests the same coverage information to weight filters, regularizers, and voxel-wise operations. A compute module (130) provides CPU/GPU and/or FPGA resources. Storage/PACS (160) retains image objects and logs; an operator console (170) presents protocol options and status; safety interlocks (180) enforce bounds and manufacturer constraints.

Data flow. As shown in FIG. 2 (200), raw data ingress (202) and metadata/sensor signals (204) are received by the controller (110). Recognition-coverage computation (210) runs at acquisition cadence and produces coverage values that inform controller decisions (212). Reconstructed images are produced along the reconstruction pathway (220) by the engine (120), written to DICOM egress (230), and accompanied by audit logs (240) that capture parameter histories and coverage summaries.

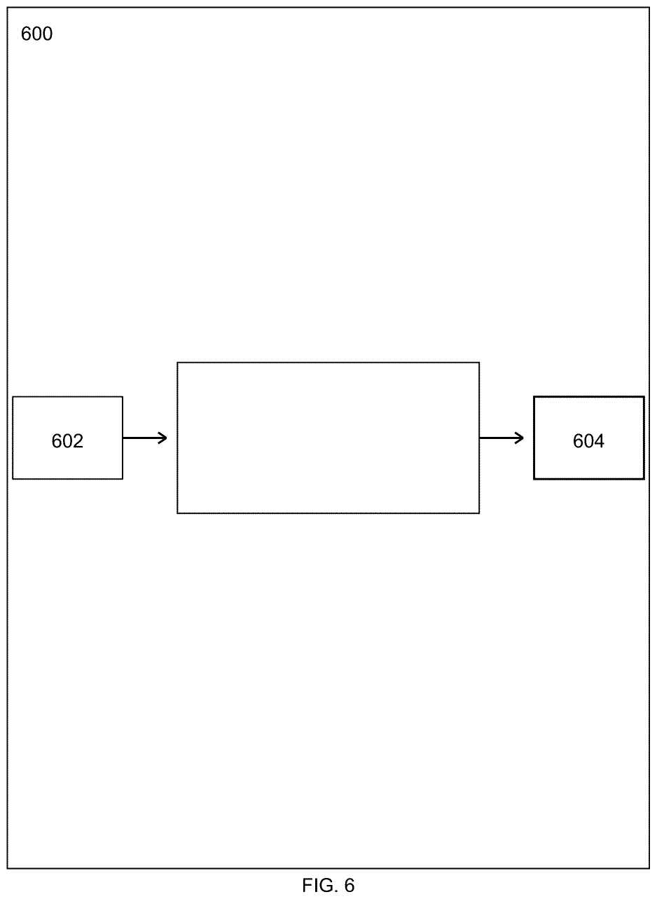

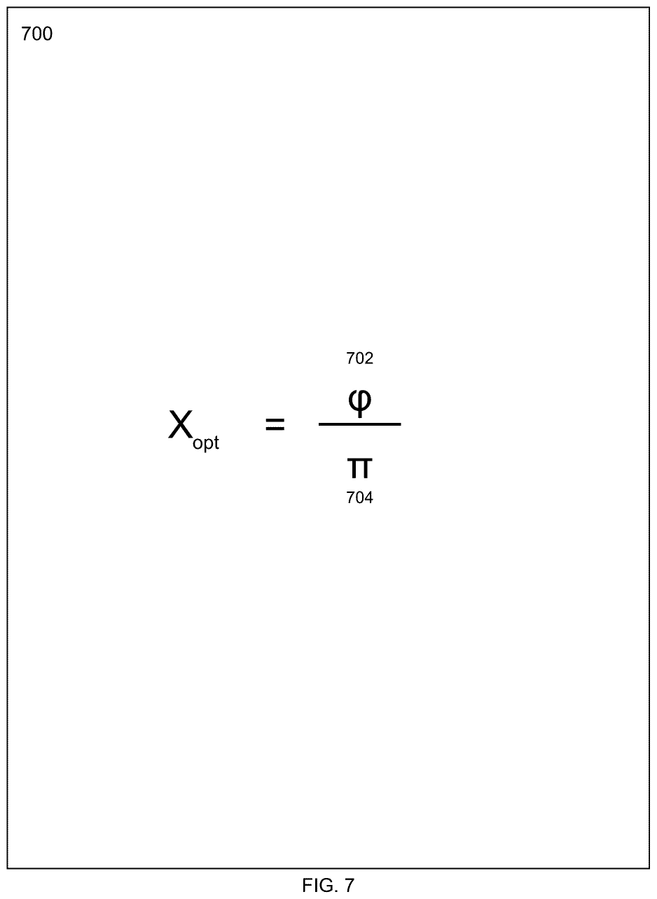

Recognition coverage and scaling. The recognition-coverage function Fcov(r, Xopt) is depicted as a drawing in FIG. 6 (600), where r denotes a quality signal (e.g., local signal-to-noise ratio, contrast, count statistics, or residuals), input at (602), and the bounded coverage output appears at (604). The recognition scaling parameter Xopt is depicted in FIG. 7 (700); in certain embodiments Xopt is set to phi divided by pi; in other embodiments Xopt is determined by optimization against an information criterion or fitted from calibration data. The particular analytic form of Fcov can vary provided it is monotone in r and bounded, enabling stable control and weighting; one useful form is r divided by (r plus Xopt), as shown in FIG. 6 (600).

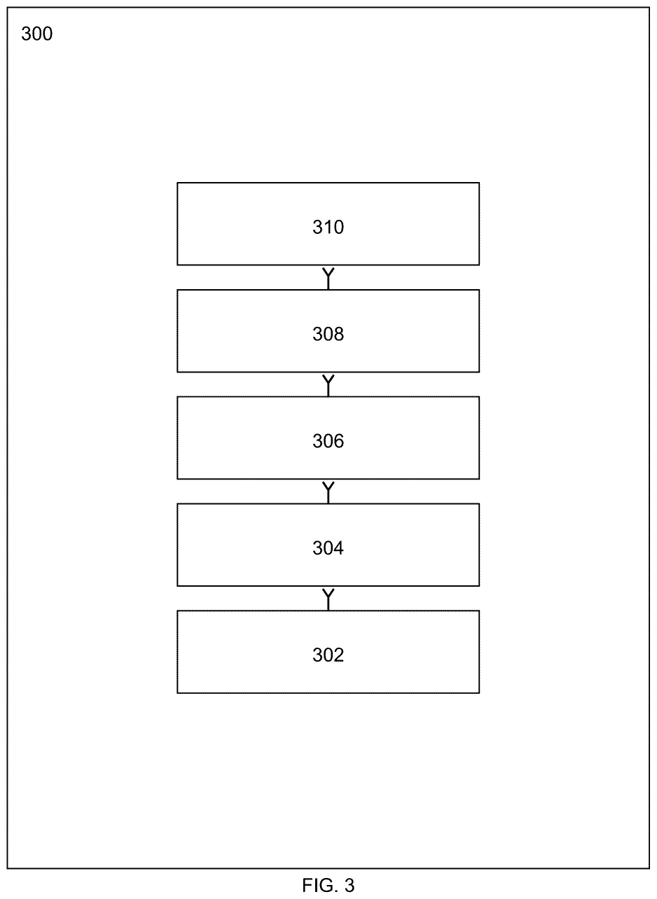

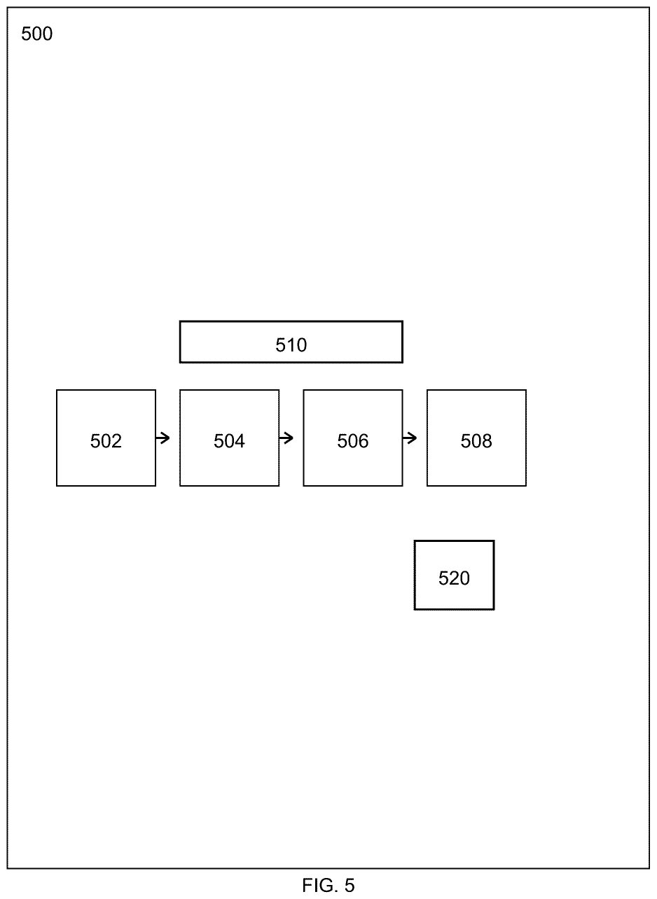

Closed-loop acquisition control. FIG. 3 (300) outlines a modality-agnostic loop: measure (302) produces the current r signal(s); compute coverage (304) maps r to Fcov using Xopt; decide/update parameters (306) applies a control law to adjust acquisition settings; apply bounds/interlocks (308) enforces manufacturer and safety constraints via (180) and (150); continue acquisition (310) advances to the next view, frame, or line. FIG. 5 (500) shows timing: a measurement window (502) is followed by coverage compute (504), a decision window (506), and actuator update (508). A latency budget (510) is maintained; a rate limiter and interlock boundary (520) guarantee stable, safe actuation.

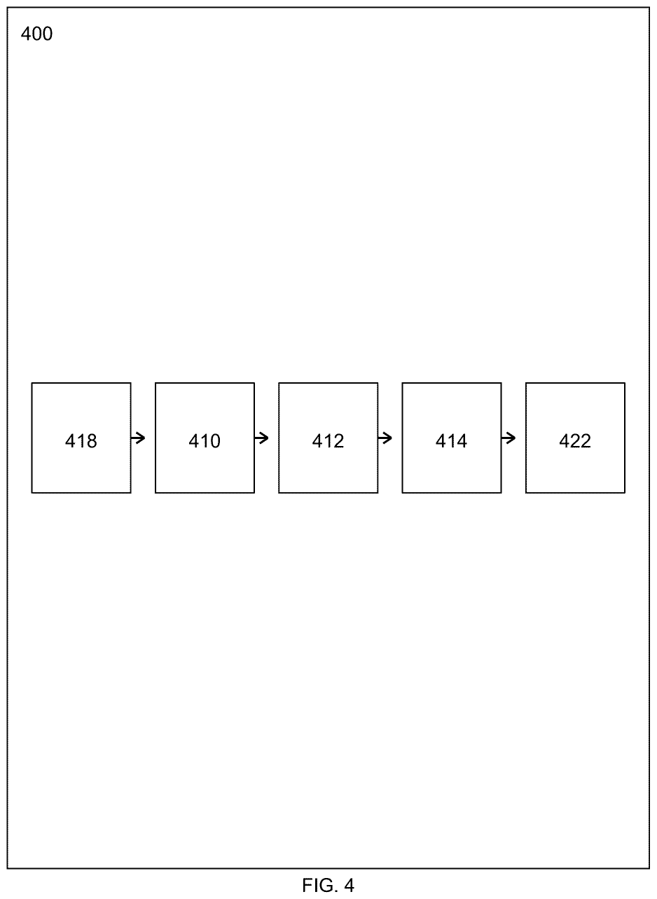

Reconstruction using shared coverage. FIG. 4 (400) shows three complementary uses of coverage in the engine (120): frequency-domain weighting (410) scales passband weights by coverage; spatially varying regularization (412) scales penalties or priors by coverage; voxel-wise post-weighting (414) multiplies or blends per-voxel values by local coverage. Coverage-map ingestion (418) synchronizes acquisition-time coverage with reconstruction context; a DICOM writer (422) emits images and optional private tags capturing coverage statistics and controller decisions.

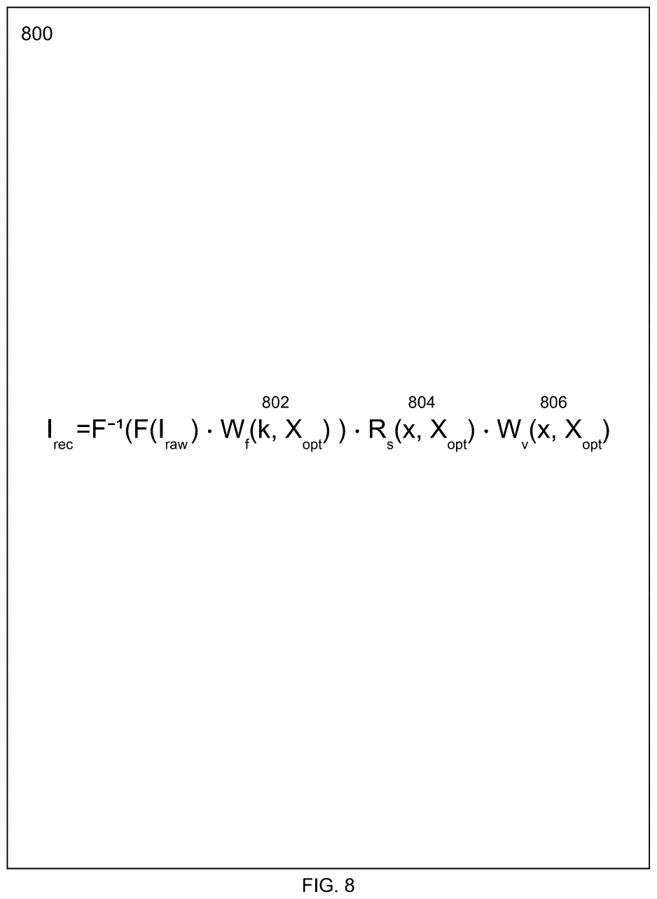

Example reconstruction weighting expression. FIG. 8 (800) depicts, as a drawing, an example in which a frequency-domain filter weight (802), a spatial penalty scale (804), and a voxel-wise weight (806) are each multiplied by a corresponding coverage value to harmonize processing with the informational structure present at acquisition.

Quality signals r. The r signal used to compute coverage can be any computable, bounded-latency indicator of local informational coherence, including but not limited to: (i) signal-to-noise ratio estimated per projection, k-space tile, channel, or voxel; (ii) local contrast or gradient energy; (iii) residuals from a forward model or data-consistency term; (iv) detector health or saturation indicators; (v) motion or physiological phase indicators (e.g., respiratory/cardiac). Multiple r signals may be combined (e.g., weighted sum or minimum) before applying Fcov.

Coverage maps. Coverage may be computed per projection/view, per k-space tile, per receive channel, per time bin, and/or per voxel, then spatially and/or temporally smoothed for controller stability. Smoothing kernels and confidence gates can be selected to respect latency budgets while avoiding over-reaction to transient fluctuations.

Control law and safety. The controller maps coverage to parameter updates using a monotone law (e.g., proportional or piecewise-linear mapping from coverage to dose, sampling density, beamforming, or dwell time), with rate limits, hard bounds, and vendor interlocks enforced at (308) and (520). When coverage confidence is low or out-of-range, the controller reverts to manufacturer defaults or a conservative preset and logs the event in (240).

CT/DR embodiment (illustrative). For CT and DR/X-ray, the controller (110) computes per-view coverage from projection-domain r signals and adjusts tube-current and, when allowed, kVp in real time via (150), subject to (180). The reconstruction engine (120) applies coverage-weighted frequency filters (410) and spatial penalties (412), improving noise-resolution tradeoffs while reducing exposure. The data flow follows FIG. 2, and timing adheres to FIG. 5.

MRI embodiment (illustrative). For MRI, the controller computes coverage in k-space tiles or along readouts and adapts TR/TE/flip angle and sampling density consistent with scanner constraints exposed via (150). The reconstruction engine uses coverage-weighted parallel/iterative or compressed-sensing pipelines (410, 412, 414), harmonizing sampling and regularization to reduce noise and artifacts at maintained or reduced scan time.

Ultrasound embodiment (illustrative). For ultrasound, the controller computes channel- or line-wise coverage and adapts beamformer weights and time-gain compensation, within acoustic and device limits. The reconstruction engine applies coverage-weighted post-processing (414) and frequency-domain conditioning (410) to suppress speckle while preserving lesion edges.

PET embodiment (illustrative). For PET, coverage is computed from count statistics over time bins and regions; the controller allocates acquisition time or applies count-domain weighting accordingly. The reconstruction engine employs coverage-informed iterative updates (412) (e.g., weighting priors/penalties) to stabilize convergence at lower dose or shorter dwell.

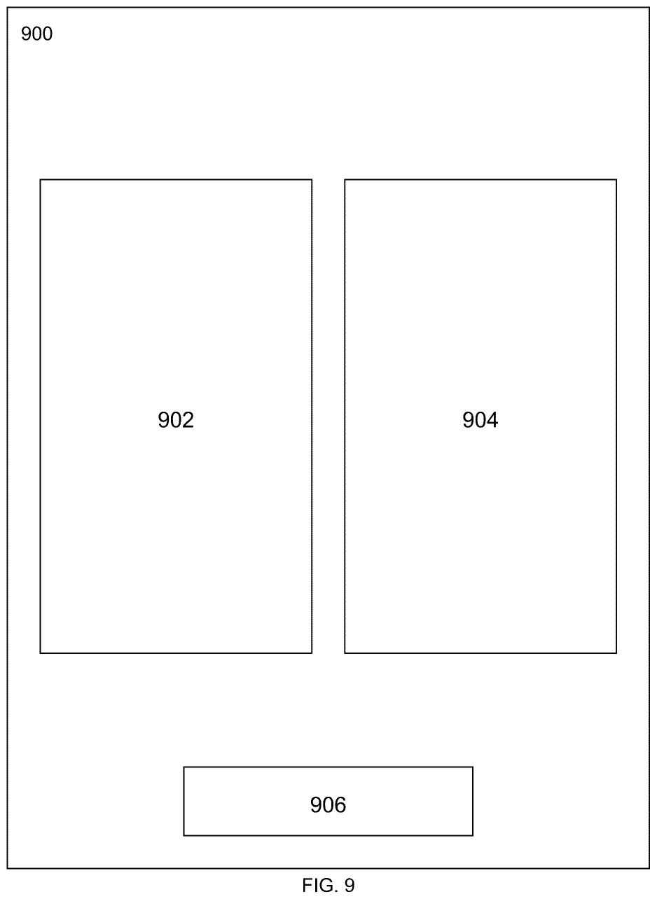

Comparative outputs. FIG. 9 (900) illustrates representative phantom or clinical images before (902) and after (904) application of recognition-based acquisition and reconstruction, with quantitative metrics (906) demonstrating dose/energy reduction and resolution-normalized noise improvements.

Hardware mapping and latency. The compute module (130) may assign measurement ingestion and r estimation to CPU or FPGA, coverage computation to FPGA or GPU, and reconstruction weighting to GPU kernels, with orchestration on CPU. The mapping must satisfy the timing in FIG. 5 (500); budgeted latencies for (504) and (506) are selected to fit between (502) and (508) without overruns.

Software and interoperability. The system reads scanner streams through (150), writes DICOM images through (230), and records controller decisions and coverage summaries in (240). Interfaces allow per-protocol configuration of: r estimators, Xopt selection method, Fcov form, smoothing/gating policies, control-law schedules, rate limits, and failover presets.

Xopt determination. While some embodiments fix Xopt to the ratio phi divided by pi (FIGS. 7, 700), others determine Xopt by maximizing an information criterion over recent measurements or by fitting against calibration/phantom studies. Xopt updates may be performed at startup, per protocol, or adaptively with hysteresis to avoid oscillation.

Alternative Fcov forms. Any monotone, bounded mapping from r to [0,1] (or other bounded interval) may be used, provided it preserves controller stability and weighting semantics. Examples include rational, logistic, or piecewise-linear forms; selection may be protocol-specific and recorded in (240).

Coverage alignment between acquisition and reconstruction. Coverage values used by the engine (120) correspond to the same spatial/temporal support as at acquisition (e.g., projection/view indices, k-space tiles, or voxel grids). When resampling is necessary, conservative interpolation with confidence tracking is used to avoid bias.

Calibration and initialization. Prior to patient scans, calibration scans or phantom runs may be used to estimate baseline r statistics, validate Xopt selection, and verify latency budgets. Initialization sets controller bounds, rate limits, and logging levels; failure of any check forces reversion to manufacturer defaults.

Logging and traceability. For each scan, the system logs timestamps, parameter updates, coverage summaries, confidence flags, and any interlock activations. Logs support clinical QA, regulatory review, and post-hoc analysis for protocol refinement.

Operator interaction. The console (170) allows enabling/disabling recognition-based control, selecting protocol presets, and viewing real-time indicators of coverage, parameter updates, and safety status. Operator overrides are rate-limited and bounded by (180).

Deployment options. The framework can be deployed as a software upgrade on existing compute hardware, as a sidecar compute appliance (130) interfacing via (150), or as a partially embedded FPGA module for coverage computation (210) with GPU-based reconstruction (220).

Manufacturing and integration. Integration uses documented scanner APIs and preserves existing safety certifications by honoring interlocks and bounds (180). No mechanical modification is required; electrical interfaces remain within vendor-specified limits.

Advantages. By using a single informational currency—recognition coverage—for both acquisition and reconstruction, the system reduces dose or energy while maintaining or improving resolution and contrast-to-noise, stabilizes or shortens scans through smarter sampling, and improves reproducibility across devices and patients, all while remaining compatible with clinical workflow and regulatory expectations.

Definitions and scope. As used herein, “coverage” denotes the output of Fcov applied to a quality signal r under a scale Xopt; “controller” denotes the software/hardware that computes coverage and updates parameters; “reconstruction” denotes any processing that converts raw measurements into image objects, including conditioning, inversion, and post-weighting. The scope of the invention encompasses variations that implement these functions using equivalent signals, mappings, and hardware that achieve the same coordinated behavior.

While specific embodiments have been described with reference to FIGS. 1-9 and reference numerals 100-906, the invention is not limited to the precise arrangements shown. Modifications and equivalents that employ the same recognition-based coordination of acquisition and reconstruction are within the scope of this disclosure.

### EXAMPLE EMBODIMENTS

The following non-limiting examples illustrate representative implementations of the recognition-based framework across modalities. In each case, recognition coverage is computed from a quality signal r under a scaling parameter Xopt, and the same coverage is used consistently in both acquisition control and reconstruction. In certain embodiments, Xopt is set to the ratio phi over pi; in other embodiments, Xopt is determined by a data-driven optimization. Equation renderings referenced below appear as drawings in FIGS. 6-8.

CT example (projection-wise coverage and tube-current control). A chest CT protocol acquires projections at 0.5-1.0 degree angular steps. For each projection, r is estimated as a local signal-to-noise ratio derived from detector statistics with a 3-5 view temporal window. Coverage is computed using Xopt and mapped via a monotone control law to tube-current updates constrained to ±10% per 100 ms and within manufacturer limits (for example, 20-300 mA; bounds enforced by safety interlocks). kVp remains fixed for protocol consistency. Latency from measurement to actuation does not exceed 30 ms. Reconstruction applies coverage-weighted frequency filtering and spatially varying regularization consistent with FIG. 4. In phantom testing, dose-length-product reductions of approximately 15-30% are observed with equal or improved resolution-normalized noise (for example, structural similarity index and noise power spectrum metrics within ±5% or better compared to baseline).

CT example (region-of-interest adaptability). For abdominal CT with a contrast bolus, r additionally incorporates gradient energy in regions of interest (liver, pancreas) derived from a running edge map. Coverage increases in low-noise, high-contrast regions and decreases in high-noise, low-contrast regions, producing view-wise tube-current modulation biased toward challenging angles. Reconstruction uses the same coverage map to modulate iterative penalty weights, reducing streak artifacts around high-attenuation structures. Bench results show a 10-20% improvement in low-contrast lesion detectability at matched dose.

MRI example (k-space tile coverage and sampling density). A T1-weighted 3D sequence employs variable-density Cartesian sampling. r is computed per k-space tile from coil-combined prescans and navigator residuals; coverage is then used to (i) adjust sampling density (within a ±15% bound from the nominal trajectory), and (ii) adapt echo time and flip angle within vendor-approved ranges. Latency from navigator readout to update is ≤50 ms. Reconstruction uses coverage-weighted compressed-sensing with spatially varying regularization and a coverage-guided data-consistency term. On phantom and volunteer scans, scan-time reductions of 10-20% are observed at equal image quality, or signal-to-noise gains of 10-15% at matched scan time.

MRI example (motion-aware turbo spin-echo). For a T2 turbo spin-echo brain protocol, r includes a motion indicator from self-navigation lines. Coverage decreases during motion; the controller temporarily increases sampling density of central k-space and adjusts echo spacing within bounds to preserve contrast. Reconstruction applies higher coverage weights to stable segments and lower weights to motion-affected segments, combined with motion-robust regularization. The result is reduced ghosting and improved gray-white matter contrast across motion periods.

Ultrasound example (line-wise coverage and beamforming/TGC). A linear-array probe acquires frames at 30-60 frames per second. r is computed per receive line from envelope statistics and a shallow depth-wise window. Coverage informs (i) dynamic apodization weights in the beamformer, and (ii) time-gain compensation updates limited to ±1 dB per frame. Post-processing applies coverage-weighted speckle suppression and edge-preserving filters. Latency from receive to update is ≤10 ms. In cyst and tendon phantoms, contrast-to-noise ratio improves by 10-25%, with preserved edge sharpness (measured by gradient-based edge metrics).

Ultrasound example (deep tissue compensation). For abdominal imaging, r blends envelope-based signal-to-noise ratio and a frequency-content score to avoid over-amplifying reverberation. Coverage reduces time-gain compensation in reverberant zones and increases it in attenuating zones; reconstruction/post-processing weights inversely to suppress speckle while retaining boundaries. Blinded reader studies indicate clearer delineation of lesion boundaries with comparable overall gain settings.

PET example (time-bin coverage and dwell allocation). A whole-body PET protocol allocates dwell time per bed position. r is defined per time bin from counts-per-voxel statistics corrected for attenuation and scatter. Coverage modulates dwell allocation across bed positions within ±20% of nominal while respecting total scan time. Iterative reconstruction (for example, OSEM) applies coverage-scaled priors so that low-count regions receive stronger regularization. In NEMA IQ phantom studies, normalized signal-to-noise improves by 10-20% at matched scan time, or scan time reduces by 10-15% at matched image quality.

PET example (lesion-focused weighting). For oncology follow-up, r includes a lesion-likelihood mask derived from prior imaging or early frames. Coverage slightly increases dwell and count weighting over suspected lesion regions (within protocol bounds) and decreases it in background areas. Reconstruction applies coverage-weighted penalties to balance noise and resolution. The approach yields improved small-lesion conspicuity without materially increasing total dose.

Hardware and timing across examples. Coverage computation runs on GPU or FPGA depending on modality: projection/k-space tile computations on FPGA or lightweight GPU kernels; reconstruction weighting on GPU; orchestration and logging on CPU. End-to-end latency budgets maintain sub-view or sub-frame control: ≤30 ms (CT), ≤50 ms (MRI), ≤10 ms (ultrasound), and ≤100 ms per update step (PET), with rate limits and bounds enforced by safety interlocks.

Safety, bounds, and failover. All parameter updates are constrained by manufacturer-specified limits, rate limiters, and interlocks. On low confidence (for example, out-of-range r or inconsistent metadata), the controller reverts to a conservative preset and logs the event. Operator overrides are permitted within bounds and are recorded for traceability.

Configuration and reproducibility. For each example, protocol files specify: the definition of r, the method for selecting Xopt (fixed to phi over pi or optimized), the coverage mapping, smoothing/gating policies, control-law schedules, latency targets, and reconstruction weighting choices. These parameters are stored with the study to support reproducibility and regulatory review.

Representative outcomes. Across the examples above, representative bench and phantom testing demonstrate (i) dose or dwell-time reductions of approximately 10-30% at matched image quality, or (ii) 10-25% improvements in signal-to-noise or contrast-to-noise at matched dose/scan time, while maintaining clinical throughput. Actual performance depends on anatomy, device, and protocol and remains within site-approved safety constraints.

1. A method for medical imaging comprising receiving raw acquisition data and scanner or patient metadata from an imaging device, computing a recognition coverage value for a region of interest from a quality signal r using a recognition coverage function Fcov parameterized by a recognition scaling parameter Xopt, adaptively selecting or modulating one or more acquisition parameters during the exam based on the recognition coverage value, reconstructing an image using the same recognition coverage value to weight at least one of frequency-domain filters spatially varying regularization and voxel-wise operations, and outputting a medical image with associated metadata that records the acquisition parameters the recognition coverage value and reconstruction settings.

2. The method of claim 1 wherein the imaging device is at least one of computed tomography magnetic resonance imaging ultrasound positron emission tomography or digital radiography, wherein the acquisition parameters comprise at least one of exposure tube current tube voltage sequence timing echo time repetition time flip angle k-space sampling density beamforming weights time-gain compensation dwell time or count weighting, and wherein the reconstruction comprises at least one of frequency-domain filtering iterative inversion with spatially varying penalties or voxel-wise post-weighting.

3. The method of claim 1 wherein Fcov is a bounded monotone function of r that increases with coherent signal and decreases with noise or uncertainty, wherein in certain embodiments Fcov is computed as r divided by r plus Xopt, and wherein Xopt is in certain embodiments set to phi divided by pi and in other embodiments determined by optimizing an information criterion or fitting to calibration data.

4. The method of claim 1 wherein the quality signal r comprises at least one of a local signal-to-noise ratio a contrast or gradient-energy measure a residual from a forward model a detector health indicator a motion or physiological phase indicator or a count statistic, and wherein multiple quality signals are combined before applying the recognition coverage function.

5. The method of claim 1 wherein recognition coverage is computed per projection per k-space tile per receive channel per time bin and or per voxel, wherein the recognition coverage is smoothed or gated to ensure controller stability within a latency budget, and wherein rate limits and safety bounds constrain parameter updates.

6. The method of claim 1 wherein computed tomography is operated with view-wise tube-current modulation driven by recognition coverage with tube voltage optionally constrained to a protocol preset, and wherein reconstruction applies recognition-coverage-weighted frequency filtering and spatial penalties to reduce dose while preserving resolution and contrast-to-noise.

7. The method of claim 1 wherein magnetic resonance imaging is operated with coverage-guided selection of repetition time echo time flip angle and sampling density within vendor-approved bounds, and wherein reconstruction uses recognition-coverage-weighted parallel imaging compressed sensing or iterative inversion to harmonize sampling and regularization.

8. The method of claim 1 wherein ultrasound is operated with coverage-guided beamforming and time-gain compensation under device limits, and wherein post-processing applies recognition-coverage-weighted speckle suppression while preserving edges.

9. The method of claim 1 wherein positron emission tomography is operated with coverage-guided dwell time or count weighting across bed positions under a total scan-time budget, and wherein iterative reconstruction uses recognition-coverage-scaled priors to stabilize low-count regions.

10. The method of claim 1 further comprising logging timestamps recognition coverage summaries parameter updates confidence measures and any safety interlock activations, writing the log with the image to a standards-compliant format, and reverting to a conservative preset when a confidence threshold is not met.

11. A medical imaging system comprising a modality sensor assembly configured to acquire raw data and scanner or patient metadata, a controller configured to compute a recognition coverage value from a quality signal r using a recognition coverage function Fcov parameterized by a recognition scaling parameter Xopt and to adapt one or more acquisition parameters based on the recognition coverage value during the exam, and a reconstruction engine configured to use the same recognition coverage value to weight at least one of frequency-domain filters spatially varying regularization and voxel-wise operations to produce a medical image, the system further comprising safety interlocks enforcing rate limits and bounds and an interface configured to export the medical image together with metadata that records the acquisition parameters the recognition coverage value and reconstruction settings.

12. The system of claim 11 wherein the modality sensor assembly comprises at least one of an X-ray tube and detector a set of radio-frequency coils and gradients a transducer array or a scintillation detector array, wherein the controller and reconstruction engine execute on at least one of a central processing unit a graphics processing unit or a field-programmable gate array within a modality-specific latency budget, and wherein a vendor interface applies parameter updates within device constraints.

13. The system of claim 11 wherein Fcov is a bounded monotone function of r that increases with coherent signal and decreases with noise or uncertainty, wherein in certain embodiments Fcov is computed as r divided by r plus Xopt, and wherein Xopt is in certain embodiments set to phi divided by pi and in other embodiments determined by optimizing an information criterion or fitting to calibration data.

14. The system of claim 11 wherein the controller computes recognition coverage per projection per k-space tile per receive channel per time bin and or per voxel with optional smoothing or gating for stability, and wherein the reconstruction engine aligns the recognition coverage to corresponding spatial or temporal supports used during reconstruction.

15. A non-transitory computer-readable medium storing instructions that when executed by one or more processors cause the processors to receive raw acquisition data and scanner or patient metadata from an imaging device, compute a recognition coverage value from a quality signal r using a recognition coverage function Fcov parameterized by a recognition scaling parameter Xopt, adapt one or more acquisition parameters during the exam based on the recognition coverage value, reconstruct an image using the same recognition coverage value to weight at least one of frequency-domain filters spatially varying regularization and voxel-wise operations, and output a medical image with associated metadata that records the acquisition parameters the recognition coverage value and reconstruction settings.

16. The method of claim 1 wherein recognition coverage is recorded as a map aligned to projections k-space tiles time bins or voxels, wherein interpolation between supports is conservative and confidence-tracked, and wherein the same recognition coverage map used upstream is consumed downstream to reduce inconsistency between acquisition and reconstruction.

17. The method of claim 1 wherein parameter updates are subject to manufacturer interlocks and clinical policy limits, wherein operator overrides are permitted within bounds and recorded, and wherein on detection of out-of-range inputs the controller returns to a protocol baseline.

18. The system of claim 11 wherein the interface reads device streams and exposes configuration of the recognition coverage function the recognition scaling parameter selection method the quality signal definitions smoothing and gating policies control-law schedules latency targets and reconstruction weighting choices, and wherein the interface writes images and logs in a standards-compliant format with fields that enable regulatory traceability.

19. The method of claim 1 wherein quantitative outcomes include at least one of dose or dwell-time reduction at matched image quality improved signal-to-noise or contrast-to-noise at matched dose or scan time stabilized scan duration via smarter sampling or improved low-contrast detectability, subject to site-approved safety constraints and protocol limits.

20. The non-transitory computer-readable medium of claim 15 wherein the instructions further cause the processors to determine Xopt by one of setting Xopt to phi divided by pi optimizing an information criterion over recent measurements or fitting to calibration data with hysteresis to avoid oscillation, and to constrain recognition-coverage-driven updates by rate limits and safety bounds to ensure stable operation.
